(19) United States
(12) Patent Application Publication
(10) Pub. No.: US 2010/0100084 A1
(43) Pub. Date: Apr. 22, 2010

(54) Hybrid Ultrafast Laser Surgery and Growth Factor Stimulation for Ultra-Precision Surgery with Healing.

(76) Inventors: Bruno Girard, Toronto (CA); Cameron Clokie, Toronto (CA); Brian Campbell Wilson, Toronto (CA); Robert John Dwayne Miller, Port Credit (CA)

Correspondence Address: MILLER THOMSON LLP, 100 STONE ROAD WEST, SUITE 301, GUELPH, ON, N1G-5L3, CA

(21) Appl. No.: 11/989,951

(22) PCT Filed: Aug. 3, 2006
(86) PCT No.: PCT/CA06/01293

§ 371 Date: Feb. 4, 2008

Related U.S. Application Data
(60) Provisional application No. 60/704,905, filed on Aug. 3, 2005.

(51) Int. Cl. A61B 18/20

(52) U.S. Cl. 606/13

(57) ABSTRACT

The present invention relates to methods for laser surgery and growth factor stimulation for ultra-precision surgery with healing. The method is achieved by cutting biological tissue using an ultrafast laser, which produces laser pulses less than 10 picosecond in duration, to induce a cold ablation process in order to avoid the formation of carbonaceous or other materials that cannot be removed efficiently or completely from the wounded area through natural healing mechanisms. By use of femtosecond lasers, a negligible amount of debris is generated and an outer layer of intact but non viable cells are created principally through shock wave induce damage and ionizing radiation effects induced by multiphoton absorption of ultrashort laser pulses. The normal healing process is blocked by this outer layer of cells as all cell contacts are still intact. Therefore the healing process must be stimulated. The healing may be triggered or accelerated, or both, by application of growth factor molecules and/or signal proteins to the effectively undamaged cells causing the damaged cells to be replaced and the wound to close. The combination of very precise laser cutting used in combination with growth factors is the key to this unique tool.

### FIELD OF THE INVENTION

The present invention relates to methods for laser surgery and growth factor stimulation for ultra-precision surgery with healing.

### BACKGROUND OF THE INVENTION

There has been a long-standing quest for more precise surgery tools to reduce healing times by allowing for more non-invasive surgery. As an example, modern surgical methods based on arthroscopic or laparoscopic procedures have reduced rehabilitation times by an order of magnitude over previous invasive procedures requiring full exposure of the injured area. The ultimate limit to precision surgery can be defined to be the scale of a single cell, about 10 microns in size. No mechanical tool can achieve this dimension with reliable accuracy. To this end, laser cutting seems like a natural technology to achieve this ultimate limit and provide the most minimally invasive procedures possible. It should be understood that throughout this disclosure, “cut” and “cutting” is used in the broad sense of cutting of biological tissue, without necessarily a line cut. Accordingly, “cut” or “cutting” as used in this disclosure also extends to, for example, ablation of biological tissue, and removal of biological tissue. There is no problem focusing laser light down to dimensions of 10 microns. With current laser technology it is no problem to deposit enough energy in such a small volume to ablate material in a highly controlled manner. The problem is that for conventional lasers (continuous-wave or long-pulsed) the ablation process requires direct absorption of the light by the biological tissues with ablation occurring through superheating. At these elevated temperatures, there is significant heat transfer to the adjacent tissue that creates collateral damage away from the targeted area. In effect, the laser burns the adjacent material creating zones of carbonized material. These blackened areas lead to improper repair of the wound, and delayed or incomplete healing.

The above problem of laser heating and destroying healthy tissue and collateral damage seemed to have been solved with the introduction of ultrafast lasers (defined to be pulsed laser systems producing pulses less than 10 picosecond in duration) for cutting through biological matter. The peak power (energy per unit time) can be enormous with such short laser pulses; yet the total energy can be maintained at very small amounts. With short enough pulses, it is possible to achieve high enough peak powers to introduce a new mechanism for ablation, in which multi-photon absorption is responsible for direct multiphoton ionization processes that lead to strong absorption within the laser focal volume, even at wavelengths where the material is nominally transparent. However, the peak power must be raised to a level that leads to the ionization of the material's constituent atoms or molecules. The liberated electrons absorb light at all wavelengths and rapidly convert this energy to heat through a known process referred to as avalanche ionization. At high enough densities, the electrons and parent ions form a plasma that can further increase the absorptivity and further localize the heat deposition. Due to the multiphoton nature of this process it is strongly dependant on the peak laser intensity, and thus the ablated volume is more localized and has edges that are much sharper than those of the laser beam's spatial profile. Thus in this short pulse limit, there is rapid localized heat deposition, leading to ablation of the superheated volume without collateral heat damage to the surrounding tissue.

Such a process for use in connection with hard tissue such as bone in “Ultrashort Pulse Lasers for Hard Tissue Ablation”, Joseph Neev et al., IEEE Journal of Selected Topics in Quantum Electronic, vol. 2, No. 4, December 1996.

This process is referred to as a “cold” ablation process. Far less heating occurs in the surrounding tissue, and the zone undergoing ablation can be reduced dramatically in spatial dimensions to the order of 10⁻¹² cm³. The result is an extremely clean cut in virtually any material with negligible or no collateral damage due to heating. U.S. Pat. No. 5,656,186 describes a laser apparatus based on this mechanism for both general laser machining and medical applications. However, the only known prior art consisting of a medical application of ultrafast laser surgery is for conducting corneal flap removal for eye surgery, where healing of the cornea is explicitly not desired. All other applications of ultrafast lasers (generally femtosecond lasers in which the laser pulses are on the order of 100 femtoseconds) have lead to deleterious collateral damage.

Despite the much-reduced heat loading in the surrounding material, there are significant collateral damage mechanisms that are unique to the ultrafast laser ablation process. There is a shock wave from the rapid ablation process and reactive ion formation upon plasma formation, which causes adjacent cell death and blocks healing. Once again it appears that laser surgery, even performed with ultrafast lasers, can only generate cutting with no healing. For eye surgery, the desired process should lead to permanent changes where healing is not desired. However, the real impact of lasers in medical surgery will not be felt until laser cutting can be accomplished with full and efficient healing of the affected tissue.

Therefore, it would be very advantageous to provide a method of cutting biological tissue using ultrafast lasers that overcomes the aforementioned problems of collateral tissue damage.

### SUMMARY OF THE INVENTION

When femtosecond lasers are used to cut biological tissues, a thin surrounding layer of mechanically intact but dead cells is created. The underlying cell-to-cell surface contacts are still in place such that the normal healing process is not stimulated. The underlying cells receive no signalling pathways to stimulate healing; the layer of dead cells, no matter how thin, blocks the healing process. In addition, the femtosecond laser cut does not produce the debris that mechanical cuts do, which suppresses the inflammatory response that naturally initiates the repair process. This thin layer of intact but dead cells must be overcome and the healing process must be stimulated. This can be achieved with growth factors molecules or signal proteins such as, for example, bone morphogenetic proteins (BMP's) that can be added to the site following wounding.

Therefore, in one aspect of the present invention a method of laser surgery is provided, the method including: a) cutting biological tissue using an ultrafast laser to form a cut so as to induce a cold ablation process in the biological tissue and thereby avoiding the formation of carbonaceous material in the biological tissue adjacent to the cut such that the biological tissue adjacent to the cut consists generally of intact but non-functioning damaged cells; and b) exposing the biological tissue adjacent to the cut to selected growth factor molecules and/or signal proteins in an amount effective to trigger and/or accelerate healing in the biological tissue adjacent to the cut, thereby promoting healing of the cut.

The ultrafast laser is preferably a femtosecond laser.

In another aspect of the present invention, the method includes the further step of selecting and applying an optimal energy range and repetition rate for a beam emitted by the femtosecond laser such that the collateral damage in the biological tissue adjacent to the cut is maintained within a range in which healing triggered and/or accelerated by the growth factor molecules and/or signal proteins is within an acceptable range.

The selected amount of growth factors and/or signal proteins is effective to overcome collateral damage in the biological tissue that results from use of femtosecond lasers. The method according to claim 3 characterized in that the effective growth factor molecules and/or signal proteins are selected from a group of molecules consisting of Bone Morphogenetic Proteins (BMPs), other members of the Transforming Growth Factors-b family (TGFs-b), Insulin-like Growth Factors (IGF-I), Colony Stimulating Factors (CSFs) and/or Epidermal Growth Factor s(EGF).

In another aspect of the present invention, an effective delivery mechanism is selected and applied for delivering the growth factor molecules and/or signal proteins to the biological tissue adjacent to the cut.

### BRIEF DESCRIPTION OF THE DRAWINGS

The methods for laser surgery and growth factor stimulation for ultra-precision surgery with healing, according to the present invention will now be described, by way of example only, reference being made to the accompanying drawings, in which:

FIG. 1a illustrates a particular instance of practice of the precise laser cutting process, namely application of the femtosecond laser beam to execute a bone bore cut. FIG. 1b further illustrates the particular instance of practice of the precise laser cutting, namely removal of the bone bore cut.

FIG. 2 in the top portion illustrates a precise laser cut in which the cells adjacent to the cut are viable and therefore receptive to healing by stimulation; in the bottom portion the cells adjacent to the cut arc carbonized due to the high temperatures generated using convention laser ablation, whereby healing is prevented.

### DETAILED DESCRIPTION OF THE INVENTION

The present invention involves a precise laser cutting process in combination with stimulation of the healing process of the mentioned layer surrounding the cut consisting of mechanically intact, dead cells that impede healing (referred to as a “non-healing layer”). The precise laser cutting process involves cutting biological material using an ultrafast laser to induce a cold ablation process, thereby avoiding the formation of carbonaceous or other materials that cannot be removed efficiently or completely from the wounded area through natural healing mechanisms.

Stimulation of healing (triggering or accelerating healing) consists of application of growth factor molecules to the non-healing layer in an amount that is effective to stimulate healing in the non-healing layer.

In the studies performed by the inventors, it has been found that it is possible to cut hone tissue with complete healing. Extremely clean cuts were observed, as had been reported previously for other types of tissue. The most significant difference was the observation of complete healing.

To the inventors' knowledge, this is the first example of ultrafast laser surgery in which full healing has been accomplished. The difference in this case is that the study focused on hard tissue materials rather than soft tissue. The shock wave propagates further in hard tissue than soft tissue. This difference means that the energy associated with the shock blast travels further before it is absorbed and the heating due to shock wave absorption will lead to much smaller temperature rises. The main cause for the general lack of healing in soft tissues with ultrafast laser cutting is the collateral damage to neighbouring cells due to the residual heating effects associated with shock wave in the ablation process and also additional mechanisms involving the high yield of reactive ions in the immediate vicinity of the cells. The key point is that the cells are not reduced to carbon in the ultrafast laser cutting process. The damaged cells can still be removed through natural healing processes and wound closure is possible. In a conventional mechanically created wound, debris is generated at the site and stimulates an inflammatory response that initiates the repair process. When femtosecond lasers are used, the neighbouring cells remain largely intact even though they are biochemically dead, so a negligible amount of debris is generated, and therefore the healing process must be externally stimulated.

This can be achieved with tissue specific growth factors molecules and/or signal proteins such as bone morphogenetic proteins (BMP's), Insulin-like Growth Factor (IGF-I), Colony Stimulating Factors (CSFs), Transforming Growth Factors-b (TGFs-b), or Epidermal Growth Factor (EGF), that can be added to the site following wounding. BMP's have been shown to accelerate new bone formation several fold. For example, “Prevention of Atrophic Nonunion Development by Recombinant Human Bone Morphogenetic Protein-7”, Takeshi Makino et al., Journal of Orthopaedic Research 23 (2005) 632-638 describes use of BMP to promote bone healing. In accordance with the present invention, growth factor molecules, such as BMP, are used to overcome the collateral damage process that is inherent to femtosecond laser ablation, by stimulating healing in the non-healing layer.

The bone studies demonstrated how close ultrafast laser cutting was to the threshold for wound healing; while simultaneously indicating the proper prescription for making the process work even for soft tissue. For bone tissue where healing was observed without BMP, the healing process was still variable and was related to the degree of damage to the surrounding tissue. Thus it is important to note in the above that the combination of the ultrafast laser cutting and growth factor molecules to initiate healing is the key enabling concept.

In a particular implementation of the present invention, wound healing was stimulated in bone by adding a BMP mixture directly to the site. Specifically, BMP-7 was combined with a 30% pluronic carrier at a concentration of 30 mg/ml, which is a reverse phase gel, which is more liquid at lower temperature and hardens as the temperature rises. The gel was applied once on the wounded site immediately after at a dose of 1.5 mg of BMP-7 per site by using a micropipette to deposit 200 ml of gel in the wound. The gel was allowed few seconds to solidify before suturing of the skin flaps. In place of BMP-7, any analogous mixture of growth factor molecules or signaling proteins in a sufficient amount to induce healing can be used, as is known in the art. In particular, the growth factors can be optimized, by those skilled in the art, to induce preferred healing in the particular tissue being cut by the femtosecond laser.

The ablation threshold in calcified bone was found to be approximately 0.7 J/cm2 when using 150 femtosecond pulses at a wavelengths around 775 nm. Cutting worked well in animal skulls up to 10 to 15 times above the ablation threshold. Above these energy densities, collateral tissue damage due to the high peak powers began to become more serious, degrading the efficiency of healing even after the addition of BMP to the wound. The threshold for ablation was found to be different in different tissues, and can be easily measured by those skilled in the art. In one aspect of the present invention therefore an optimal energy range and repetition rate is determined for the particular precise cutting operation such that the collateral tissue damage is maintained at levels where stimulated healing is acceptable, in a manner that is known to those skilled in the art.

A Ti:Sapphire regenerative amplifier system producing 0.2 mJ, 150 fs pulses at a wavelength near 775 nm at a repetition rate up to 1 kHz was used to perform bone surgery. The method can also make use of any other laser known to those skilled in the art that produces pulses shorter than about 10 ps at any wavelength, with enough energy per pulse to achieve the threshold for ‘cold ablation’ in the tissue of interest. Such lasers include but are not limited to, amplified Ti:Sapphire laser systems, amplified Er:glass fiber lasers, amplified Yb:glass fiber lasers, amplified Cr:Forsterite laser systems, and the amplified output of solid-state mode-locked laser oscillators such as YLF, YAG and Vanadate.

The femtosecond pulses from the laser can be delivered using any convenient optical fibers known to those skilled in the art that are able to support the wavelength and peak power of the pulses. The fiber can be terminated by lenses and/or other optical components that produce the desired laser focal conditions at the location of the tissue to be ablated, or the light can be used to ablate tissue placed directly at the output of the fiber. The end of the fiber can be mounted in a hand-held handle for direct use by the surgeon, it can be mounted on an endoscopic device for non-invasive internal surgery, or to achieve the maximum possible precision of cutting it can be held by a robotic surgery device.

The present invention is illustrated with respect to the following non-limiting examples.

### Example 1

Laser Cutting in the 10 Micron Range

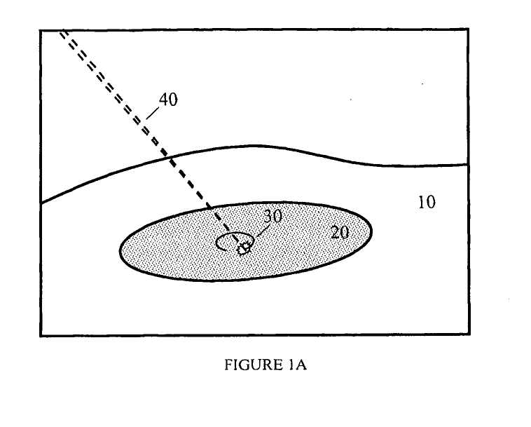

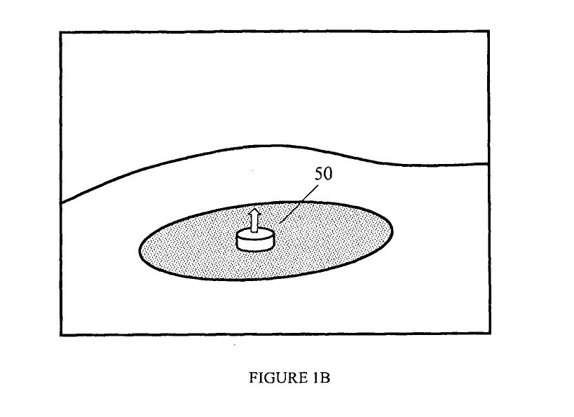

A demonstration that femtosecond lasers can ablate osseous tissues with micron precision, far exceeding the precision of conventional rotary mechanical instruments, is shown in FIGS. 1a and 1b. In surgery, smaller cuts are favorable over wider cuts since healing will occur more rapidly and completely.

FIGS. 1a and 1b show a circular cut 30 created in the head 10 of a mouse with a femtosecond laser 40 in mouse calvaria 20. A core 50 can be removed, as shown in FIG. 1b. The cut width is smaller than any cut possible with a mechanical device. In fact, with mechanical instruments the smallest possible cut width is even larger than the entire core created using the laser femtosecond laser. Cores smaller than 2 mm in diameter are generally not possible with mechanical instrumentation. Also note the irregular border of the osteomtomy.

The cut shown in FIGS. 1a and 1b has a width of approximately 50 um, approximately 20 times smaller than a small mechanical cut. In comparison, a small mechanical instrument would remove the entire circle (approx 1.2 mm). No visible debris/fragments are created during the laser cut. Mechanical instruments generate bone particles that must be washed away from the site because they can lead to acute inflammatory responses, slower wound healing and infections.

### Example 2

Healing

Studies were conducted to compare the healing of femtosecond laser created wounds to mechanically caused ones. The wounds are made on a 4 week-old mouse calvaria, bilaterally with either mechanical diamondor carbide rotary bur or a femtosecond laser. In this particular study the wounds created were critical size, meaning that they were too large to spontaneously heal. It was found that all wounds healed at a similar rate. Using growth factor molecules, wounds are completely closed at the 12 week period demonstrating that there is no detrimental effect from the laser irradiation on growth factor molecule induced healing. What is most important here is that since the cuts performed by the laser are 20 times smaller then any mechanical cuts, healing triggered by BMP, for example, would lead to healing faster than any mechanical instruments. For the specific case of bone tissue, this invention will cover the BMP's that induce healing and accelerate bone formation.

The combination of very small cuts and growth factor molecules is a unique combination that leads to faster wound healing than can be achieved with current technology.

### Example 3

Demonstration of Laser Precision Limits at Single Cell Boundaries.

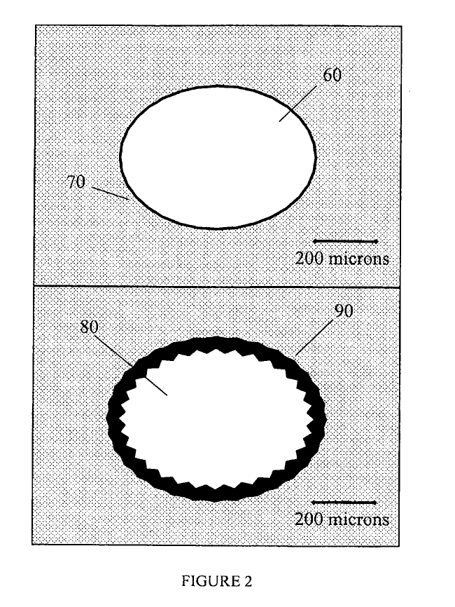

FIG. 2 shows microphotographs of excised mouse calvaria after laser irradiation (top) Ultrafast Laser: λ=775 nm 100 μJ/pulse, 1 kHz, τ=150 fs, Alkaline phosphatase (AP) staining (blue colour) is active on the cell surface in the area immediately adjacent to ablation. (Bottom) Conventional Laser: λ=535 nm τ=150 ns 1 mJ/pulse, 1 kHz, showing charring in the wound periphery: AP has been denatured up to 200 μm from the ablated area, indicating a temperature rise above 56° C. The brown circular area is charred tissue that impairs healing, comprised of carbonized matter produced at the high temperatures generated during ablation with conventional lasers. In the top portion there is cell viability immediately adjacent to the laser wound.

As illustrated by the results, the precision achieved with femtosecond lasers also outperforms conventional lasers, since the tissues do not appear to suffer any thermal damage as seen with in vitro testing showing intact enzymatic activity on cells immediately adjacent to wounding. The degree of variable healing when growth factor molecules are not used is related to the laser induced damage to this zone immediately adjacent to the cut.

In summary, the present invention provides a surgical method where an ultrafast laser removes a single cell or region of cells, causing partial damage to a single or several surrounding layers of cells and causes effectively no damage to cells beyond. The partial damage is limited to factors affecting the viability of the specific cells, and excludes formation of carbonaceous materials that would limit the healing of the treated area. The natural healing process may be triggered in the effectively undamaged cells causing any damaged cells to be replaced and the wound to close. The healing may be triggered or accelerated, or both, by application of growth factor molecules and/or signal proteins to the effectively undamaged cells causing the damaged cells to be replaced and the wound to close.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

There are a number of possible applications of the present invention. These include: (A) application to bone reconstructive surgery, for example, use of the method of the present invention to cut a bone plug in a specific desired shape from a donor site, and to prepare a receptor site for the donor bone plug; (B) bone surgery, for example, any osteotomy required for fracture management or osteomies necessary for management of skeletal deformities; (C) use for surgery in other tissues: for example site preparation in liver, heart bypass, kidney, pancreas and brain surgery; (D) non-invasive internal surgery: for example use for minimally invasive surgery on internal organs and tissue, when combined with endoscopic techniques known to those skilled in the arts; (E) cosmetic surgery: use to make thin cuts with minimal scarring in cosmetic surgeries.

Therefore what is claimed is:

1. A method of laser surgery, characterized in that the method includes:
a) cutting biological tissue using an ultrafast laser to form a cut so as to induce a cold ablation process in the biological tissue and thereby avoiding the formation of carbonaceous material in the biological tissue adjacent to the cut such that the biological tissue adjacent to the cut consists generally of intact but non-functioning damaged cells; and
b) exposing the biological tissue adjacent to the cut to selected growth factor molecules and/or signal proteins in an amount effective to trigger and/or accelerate healing in the biological tissue adjacent to the cut, thereby promoting healing of the cut.

2. The method of claim 1, characterized in that the ultrafast laser consists of a femtosecond laser.

3. The method of claim 1, characterized in that the growth factor molecules and/or signal proteins are matched to the biological tissue.

4. The method of claim 2, characterized in that method includes the further step of selecting and applying an optimal energy range and repetition rate for a beam emitted by the femtosecond laser such that the collateral damage in the biological tissue adjacent to the cut is maintained within a range in which healing triggered and/or accelerated by the growth factor molecules and/or signal proteins is within an acceptable range.

5. The method of claim 2, characterized in that the selected amount of growth factors and/or signal proteins is effective to overcome collateral damage in the biological tissue that results from use of femtosecond lasers.

6. The method according to claim 3 characterized in that the effective growth factor molecules and/or signal proteins are selected from a group of molecules consisting of Bone Morphogenetic Proteins (BMPs), other members of the Transforming Growth Factors-b family (TGFs-b), Insulin-like Growth Factors (IGF-I), Colony Stimulating Factors (CSFs) and/or Epidermal Growth Factor s(EGF).

7. The method of claim 1 characterized in that an effective delivery mechanism is selected and applied for delivering the growth factor molecules and/or signal proteins to the biological tissue adjacent to the cut.

8. The method of claim 1 characterized in that the biological tissue consists of bone and the growth factor molecules consist of BMPs.

9. The method of claim 1 characterized in that the biological tissue consists of soft tissue and said growth factor molecules and/or signal proteins are matched to the type of tissue being cut.
